(19) United States
(12) Patent Application Publication
(10) Pub. No.: US 2006/0030780 A1
(43) Pub. Date: Feb. 9, 2006

(54) System and method providing controllable attenuation of an ultrasound probe

(76) Inventors: Jean-Francois Gelly, Valbonne (FR); Steven Charles Miller, Pewaukee, WI (US)

Correspondence Address: DEAN D. SMALL;ARMSTRONG TEASDALE LLP, ONE METROPOLITAN SUITE 2600, ST. LOUIS, MO, 63102, US

(21) Appl. No.: 10/910,968

(22) Filed: Aug. 3, 2004

(51) Int. Cl. A61B 8/14

(52) U.S. Cl. 600/459

(57) ABSTRACT

Systems and methods for providing controllable attenuation of an ultrasound probe are provided. The ultrasound probe includes a housing having a membrane, a transducer array within the housing and a fluid between the transducer array and the membrane. The membrane is configured to contact an object and the fluid is configured to allow controllable attenuation of the ultrasound probe.

### BACKGROUND OF THE INVENTION

This invention relates generally to ultrasound systems. More particularly, the invention relates to systems and methods for providing controllable attenuation of an ultrasound probe.

Ultrasound systems typically include a probe with a transducer array that generates acoustic waves and receives the echoes of these waves. The echoes may be reflected, for example, by a part of human body under examination. The reflected echoes include scanning information related to the object under examination. Further, the ultrasound systems typically include a processor that performs processing of the received scanning information. A display generates images from the ultrasound scanning information processed by the processor.

Known ultrasound systems may have image alteration caused by various artifacts. These artifacts may result from echoes received as part of scanning information that do not correspond in location or intensity to actual interfaces in the body under examination. These artifacts are undesirable because they interfere with the interpretation of the images.

Images that are produced with transducer arrays commonly have reverberation artifacts. These artifacts are caused by secondary ultrasound emissions produced from the reflections of echoes from the face of the transducer array in contact with the body of the patient. The reverberation artifacts need to be attenuated in order to generate clear and distortion-free images. However, this attenuation also results in the attenuation of the received scanning information (e.g., loss of signal to noise in the signal processing chain).

Further, increasing the intensity of the acoustic output to enhance the received echoes causes heating up of the transducer array surface, thereby limiting the amount of possible enhancement. In particular, the face of the transducer probe heats up during normal scanning due to absorption of sound in the acoustical lens material. Therefore, ultrasound systems must limit the acoustic output to avoid the risk of, for example, burning patients. This also may be necessary in order to comply with mandated acoustical lens temperature guidelines.

Thus, known ultrasound systems may not adequately provide controllable attenuation of an ultrasound probe. The known ultrasound systems may cause uncontrolled reverberation and/or attenuation of received scanning information or excessive heating of the transducer probe with the attenuation methods implemented.

### BRIEF DESCRIPTION OF THE INVENTION

In one embodiment, an ultrasound probe is provided. The ultrasound probe includes a housing having a membrane, a transducer array within the housing and a fluid between the transducer array and the membrane. The membrane is configured to contact an object and the fluid is configured to allow controllable attenuation of the ultrasound probe.

In another embodiment, a method for controlling an ultrasound system is provided. The method includes providing a liquid within an ultrasound probe between a transducer array and a surface for contacting an object. The method further includes configuring the liquid to allow controllable attenuation of the ultrasound probe.

### BRIEF DESCRIPTION OF THE DRAWINGS

FIG. 1 is a block diagram illustrating an ultrasound operational environment of various embodiments of the invention.

FIG. 2 is a block diagram of a portion of ultrasound probe in accordance with various embodiments of the invention.

FIG. 3 are block diagrams illustrating the operation of an ultrasound probe in accordance with various embodiments of the invention.

FIG. 4 is a flowchart illustrating a method for controlling an ultrasound system in accordance with an embodiment of the invention.

### DETAILED DESCRIPTION OF THE INVENTION

Various embodiments of the invention provide methods and systems for allowing controllable attenuation of an ultrasound probe.

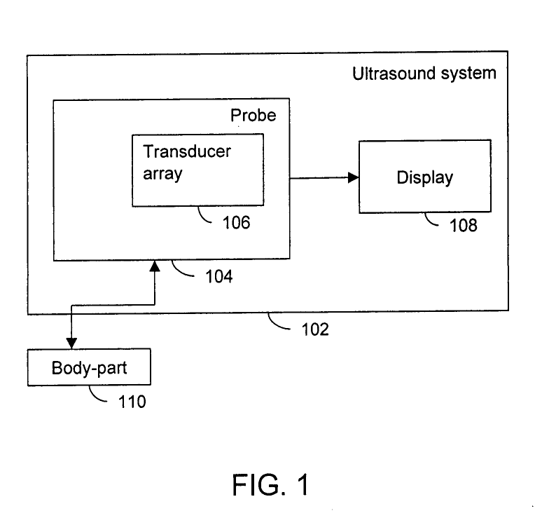

FIG. 1 is a block diagram illustrating an ultrasound operational environment of various embodiments of the invention. Ultrasound system 102 includes an ultrasound probe 104 having a transducer array 106 and a display 108. Transducer array 106 may include an acoustical lens covering at least part of transducer 106. Transducer array 106 within ultrasound probe 104 generates acoustic waves and receives the echoes as reflected by an object, such as, for example, a body part 110 of a patient under examination. The reflected echoes constitute scanning information about the body part under examination. A part of the reflected echoes is reflected from the surface of transducer array 106 back to the patient, which may result in the formation of reverberation artifacts. Transducer array 106 may use piezoelectric or electrostatic effect to communicate the scanning information regarding body part 110 to display 108, which displays images based on the scanning information.

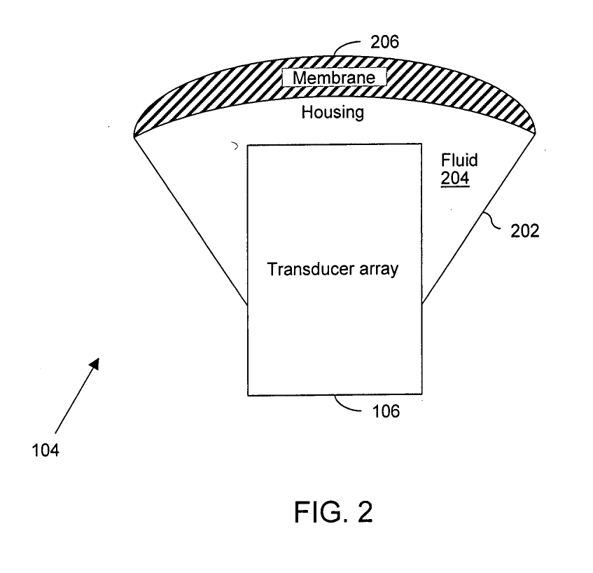

FIG. 2 is a block diagram of a portion of an ultrasound probe 104 in accordance with various embodiments of the invention. Ultrasound probe 104 (shown in FIG. 1) includes a housing 202, a transducer array 106 and a fluid 204 within the housing 202. In various embodiments of the invention, the geometry of housing 202 may be, for example, toroidal, cylindrical, spherical or flat. Housing 202 is adhered or attached to ultrasound probe 104 using, for example, glues such as silicon or epoxy glues. Housing 202 further includes a membrane 206. Membrane 206 may be integrally formed with the housing 202 or separately attached thereto. Housing 202 further includes a sealed portion for maintaining a fluid-tight seal between fluid 204 and transducer array 106. Exemplary materials suitable for housing 202 include thermoplastic materials, but can also be made with the same material as the membrane.

Membrane 206 is configured to contact an object. In one exemplary embodiment membrane 206 may be constructed of a soft material that has an impedance and viscosity close to water. Exemplary materials suitable for membrane 206 include, for example, Conap-urethane EN-4, rubber, rencothane 6400, polyurethane, neoprene, polybutadene, polycarbonate syloxane, methylpentene coplymers such as TPX®. TPX® is a 4-methylpentene-1 available from Mitsui Chemicals, Inc. etc. Membrane 206 may also be an electro-active material with attenuation properties electrically controlled. Membrane 206 is adhered on the edges of housing 202 in one embodiment. The thickness of membrane 206 generally ranges from about 0.5 millimeters to about 2 millimeters. The object contacted may be body part 110 (shown in FIG. 1), which is under examination. Membrane 206 is generally pliable. In particular, membrane 206 is not rigid, and may be readily deformed upon application of pressure (e.g., mechanical or manual application of pressure). When membrane 206 is placed on body part 110, membrane 206 deforms and conforms to the shape of the contact surface. This results in increased patient comfort during examination.

Transducer array 106 may be a mechanically controlled transducer array or an electronically controlled transducer array. For example, a mechanically steerable transducer array 106 may be provided in a wet chamber separated from a dry chamber having control components therein. As another example, transducer array 106 may be fixed with the elements selectively operable. The various embodiments, however, are not limited to a particular probe type, but may be implemented in any type of probe.

Fluid 204 is an acoustic fluid between transducer array 106 and membrane 206 and is configured to allow controllable attenuation of ultrasound probe 104. The acoustic impedance of fluid 204 is similar to the acoustic impedance of human tissues. This minimizes the scattering of the ultrasound wave. Further, in various embodiments of the invention, fluid 204 may be a resilient material having an acoustic impedance and viscosity similar to acoustic fluids. Fluid 204 may also be an electro-active material with attenuation properties electrically controlled.

In various embodiments of the invention, the attenuation of ultrasound probe 104 (shown in FIG. 1) is controlled based upon the pressure applied to membrane 206. The pressure applied to membrane 206 may be hand pressure applied by a user holding ultrasound probe 104. The level of attenuation is generally between about zero decibels and about thirty decibels, and for example, between about five decibels and about fifteen decibels. However, depending on the type of ultrasound probe 104 and application, the range of attenuation may be configured to vary accordingly. In addition, the level of attenuation may be controlled mechanically.

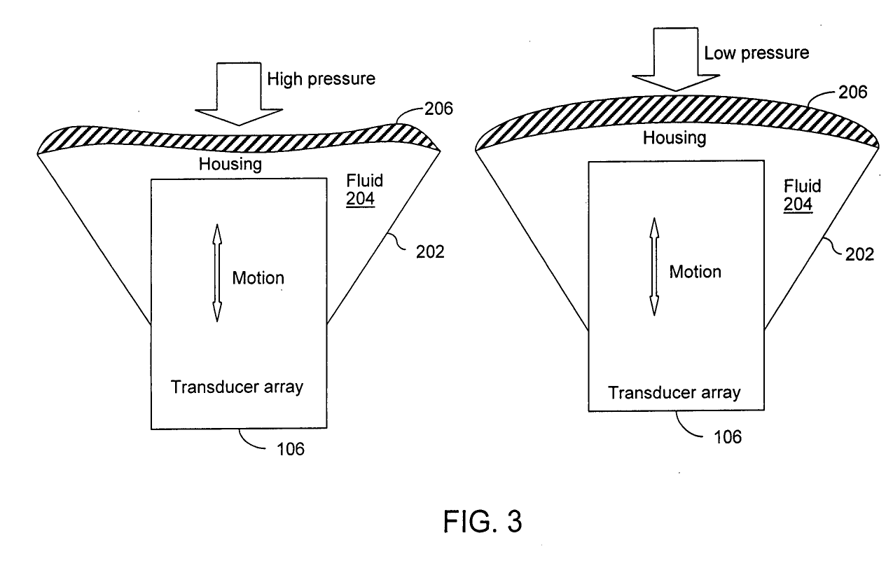

FIG. 3 illustrates the operation of a probe in accordance with an exemplary embodiment of the invention. When a varying pressure is applied to membrane 206 the volume of fluid 204 in different regions between housing 202 and membrane 206 and/or transducer array 106 varies. The pressure may be varied manually by a user applying different pressure (e.g., hand pressure) to the ultrasound probe 104 that is in contact with an object, thereby changing the pressure applied to membrane 206. The pressure also may be varied mechanically, for example, transducer array 106 may be moved mechanically within ultrasound probe 104 (shown in FIG. 1) thereby changing the pressure on membrane 206. The mechanical movement of transducer array 106 may be provided, for example, by a sliding movement using a motor arrangement. Consequently, the thickness of fluid 204 between membrane 206 and transducer array 106 varies. The higher the applied pressure, the greater the reduction in thickness of the fluid layer and vice versa. The thickness of the fluid layer in one embodiment may vary from about zero to about three millimeters or more. However, depending on the type and configuration of ultrasound probe 104, the amount and type of fluid 204 may be modified to allow for different ranges of thicknesses. Further, the echoes that are reflected back into the patient are partly absorbed by fluid 204. Therefore, a reduction in reverberation artifacts, which may cause distortions in the image, is provided. It should be noted that ultrasound probe 104 may include in various embodiments a fluid reservoir (not shown), for example, an elastic or balloon like structure to store fluid 204 when the volume of fluid 204 is compressed. For example, a two way valve or similar structure may provide for passage of fluid 204 into and out of a fluid reservoir depending on the pressure being applied. In other embodiments, the configuration or shape of ultrasound probe 104 may be modified, for example, providing additional space to accommodate fluid 204 when compressed.

The absorption capacity of fluid 204 varies in accordance with the volume of fluid 204 between membrane 206 and transducer array 106 in housing 202. As the volume of fluid 204 increases, the absorption capacity of fluid 204 also increases. Therefore, with variation in pressure to membrane 206, the absorption capacity of fluid 204 varies. Consequently, the pressure is applied based on, for example, the desired degree of reduction or elimination of reverberation artifacts within an image on display 108 (shown in FIG. 1) relating to an object being scanned. Further, with a change in thickness of fluid 204 between membrane 206 and transducer array 106 the acoustic path length varies through the attenuating medium. Therefore, fluid 204 is configured to provide reverberation reduction characteristics based on operating parameters of transducer array 106. Thus, based upon an image being displayed, and more particularly, the quality of the image desired or features to be observed, adjustable attenuation of, for example, artifacts or other distortions (e.g., superimposed images), is provided.

In various embodiments of the invention, the desired degree of reduction or elimination of reverberation artifacts within an image displayed on display 108 (shown in FIG. 1) relating to an object being scanned may be provided by using fluid 204 that has high level attenuation, specifically about fifteen decibels and adjusting the thickness of fluid 204 between membrane 206 and transducer array 106 (shown in FIG. 1) to smaller values, specifically between zero and one millimeters, as described herein.

Fluid 204 further reduces the heat generated within housing 202. As the acoustical lens temperature of transducer array 106 (shown in FIG. 1) increases, fluid 204 cools the acoustical lens temperature through convection and/or fluid forced circulation. Consequently, ultrasound probe 104 (shown in FIG. 1) may be operated at higher intensities of the ultrasound wave, thereby improving the sensitivity of ultrasound probe 104. Fluid 204 between transducer array 106 and membrane 206 allows membrane 206 to have a lower temperature than the acoustical lens of transducer array 106. This enables operation of ultrasound probe 104 at higher intensities, even when acoustical lens temperature is higher than the mandated guidelines (e.g. IEC 60601-2-37 acoustical lens temperature requirements). This is because the patient is in contact with membrane 206 rather than the acoustical lens of transducer array 106. Fluid 204 is, therefore, configured to provide heat transfer characteristics based on operating parameters of transducer array 106. The operating parameters may be, for example, the intensity of ultrasound wave, acoustical lens temperature, etc. It should be noted that the fluid cooling effect contributes to the effectiveness of the attenuation control as it allows an increase in the transmitted power and also compensates the signal to noise loss resulting from the fluid attenuation.

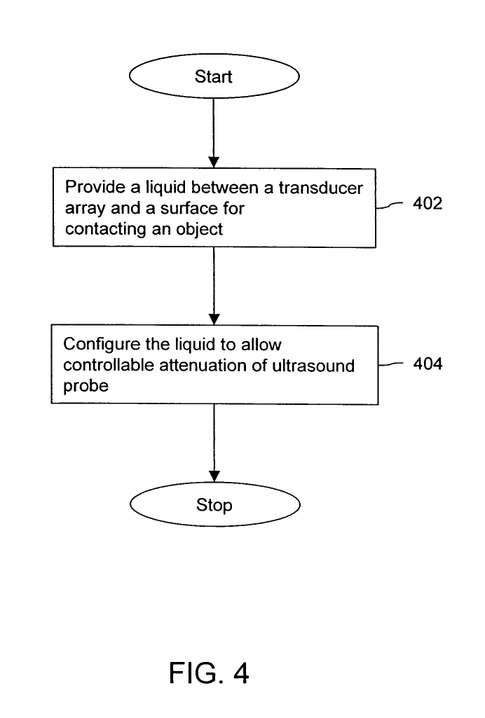

FIG. 4 is a flowchart illustrating a process for controlling an ultrasound system in accordance with an embodiment of the invention. At 402, a liquid is provided between transducer array 106 (shown in FIG. 1) and a surface for contacting an object. The liquid may be fluid 204 (shown in FIG. 1) and the surface may be membrane 206 (shown in FIG. 2). Thereafter, the liquid is configured to allow controllable attenuation of ultrasound probe 104 (shown in FIG. 1) at 404. The liquid further may be configured to reduce heat generated within ultrasound probe 104. The level of attenuation can be controlled by a user input. The user input may include, for example, manual pressure to ultrasound probe 104 and/or a control command to control mechanically the level of attenuation.

The various embodiments of the invention provide an ultrasound probe having controllable attenuation. Further, the various embodiments of the invention allow a controllable reduction of reverberation artifacts. The user may easily balance the degree of attenuation and reverberation artifact reduction required by varying the pressure applied to the ultrasound probe. The various embodiments of the invention also improve sensitivity of the ultrasound probe by preventing heating of the patient contact surface. This further enables compliance with certain mandated guidelines (e.g., IEC 60601-2-37 acoustical lens temperature requirements). Additionally, the various embodiments of the invention increase patient comfort levels by using a pliable membrane, which when placed on the body part under examination, deforms and conforms to the body part.

While the invention has been described in terms of various specific embodiments, those skilled in the art will recognize that the invention can be practiced with modification within the spirit and scope of the claims.

What is claimed is:

1. An ultrasound probe comprising:
a housing having a membrane configured to contact an object;
a transducer array within the housing; and
a fluid between the transducer array and the membrane, the fluid configured to allow controllable attenuation of the ultrasound probe.

2. An ultrasound probe in accordance with claim 1 further comprising an acoustical lens covering at least part of the transducer and wherein the fluid is between the acoustical lens and the membrane.

3. An ultrasound probe in accordance with claim 1 wherein the fluid is configured to reduce temperature of a probe front face.

4. An ultrasound probe in accordance with claim 1 wherein the transducer array is a mechanically controlled array.

5. An ultrasound probe in accordance with claim 1 wherein the transducer array is an electrically controlled array.

6. An ultrasound probe in accordance with claim 1 wherein a level of attenuation is based upon a pressure applied to the membrane.

7. An ultrasound probe in accordance with claim 6 wherein the pressure is hand pressure applied by a user holding the probe.

8. An ultrasound probe in accordance with claim 6 wherein the pressure is mechanically applied.

9. An ultrasound probe in accordance with claim 6 wherein the pressure applied is based on an image on an ultrasound display relating to an object being scanned with the transducer array.

10. An ultrasound probe in accordance with claim 9 wherein the pressure applied is based on a desired reduction in reverberations causing distortions to the image.

11. An ultrasound probe in accordance with claim 6 wherein the pressure applied is based on a user viewing an artifact within an image on an ultrasound display relating to an object being scanned with the transducer array.

12. An ultrasound probe in accordance with claim 1 wherein the fluid comprises an acoustic fluid having an acoustic impedance about the same as an acoustic impedance of human tissue.

13. An ultrasound probe in accordance with claim 1 wherein the fluid is configured to provide heat transfer characteristics based on operating parameters of the transducer array.

14. An ultrasound probe in accordance with claim 1 wherein the fluid is configured to provide reverberation reduction characteristics based on operating parameters of the transducer array.

15. An ultrasound probe in accordance with claim 1 wherein the fluid is configured to provide attenuation characteristics based on operating parameters of the transducer array.

16. An ultrasound probe in accordance with claim 1 further comprising a sealed portion within the housing for maintaining a liquid tight seal between the liquid and an inside of the housing.

17. An ultrasound probe in accordance with claim 1 wherein a level of attenuation is based upon a thickness of the fluid.

18. An ultrasound probe in accordance with claim 17 wherein the thickness is between about zero millimeters and about three millimeters.

19. An ultrasound probe in accordance with claim 1 wherein a level of attenuation is based upon a volume level of the fluid.

20. An ultrasound probe in accordance with claim 1 wherein a level of attenuation is controlled mechanically.

21. An ultrasound probe in accordance with claim 1 wherein a level of attenuation is controlled manually by a user applying pressure to the membrane.

22. An ultrasound probe in accordance with claim 1 wherein a level of attenuation is between about five decibels and about fifteen decibels.

23. An ultrasound system comprising:
an ultrasound scanning system having a probe for scanning a patient; and
a display for displaying images generated from an ultrasound scan of the patient using the probe, the probe having a fluid therein, the fluid configured to allow for controlling attenuation of the probe based upon distortions in an image displayed on the display.

24. An ultrasound system in accordance with claim 23 wherein a level of attenuation is controlled manually by a user.

25. An ultrasound system in accordance with claim 23 wherein a level of attenuation is controlled mechanically.

26. An ultrasound system in accordance with claim 23 wherein the fluid is configured to reduce heat generated within the probe.

27. An ultrasound system in accordance with claim 23 wherein the probe comprises a mechanically controlled transducer array.

28. An ultrasound system in accordance with claim 23 wherein the probe comprises an electrically controlled transducer array.

29. A method for controlling an ultrasound system, said method comprising:
providing a liquid within an ultrasound probe between a transducer array and a surface for contacting an object; and
configuring the liquid to allow controllable attenuation of the probe.

30. A method in accordance with claim 29 further comprising configuring the liquid to reduce heat generated within the probe.

31. A method in accordance with claim 29 further comprising receiving a user input to control a level of attenuation.

32. A method in accordance with claim 31 wherein the user input comprises manual pressure applied to the probe to control the level of attenuation.

33. A method in accordance with claim 31 wherein the user input comprises a control command to control mechanically the level of attenuation.

34. A method in accordance with claim 29 wherein the probe comprises one of a mechanically controlled transducer array probe and an electrically controlled transducer array probe.
